SEQUENCE LISTING (2 sequences)

SEQ ID NO: 1 - DNA, 20 bp, mice
aagagcatttttcaaaaata

SEQ ID NO: 2 - DNA, 20 bp, mice
gtctcagaatttgctttatt

(19) United States
(12) Patent Application Publication
(10) Pub. No.: US 2011/0082103 A1
(43) Pub. Date: Apr. 7, 2011

(54) Anoectochilus SPP. polysaccharide extracts for stimulating growth of advantageous bacteria, stimuating release of granulocyte colony-Stimulating factor, modulating T helper cell type I, and/or modulating T helper cell type II and uses of the same

(75) Inventors: Wen-Chuan Lin, Taichung (TW); Chang-Chi Hsieh, Taichung (TW); Ting-Jang Lu, Taichung (TW); Hsin-Sheng Tsay, Taichung (TW); Li-Chan Yang, Taichung (TW); Chia-Chieh Lin, Taichung (TW); Chung-Huang Wang, Taichung (TW)

(73) Assignee: CHINA MEDICAL UNIVERSITY, Taichung (TW)

(21) Appl. No.: 12/660,580

(22) Filed: Mar. 1, 2010

(30) Foreign Application Priority Data
Oct. 5, 2009 (TW) 098133714

(51) Int. Cl. A61K 31/715; A61P 1/00; A61P 3/14; A61P 29/00; A61P 37/00; A61P 35/00; A61P 11/06

(52) U.S. Cl. 514/54

(57) ABSTRACT

An Anoectochilus spp. polysaccharide extract for stimulating the growth of advantageous bacteria, stimulating the release of granulocyte colony-stimulating factor (G-CSF), modulating T helper cell type I (Th1 cell), and/or modulating T helper cell type II (Th2 cell) is provided. The extract comprises an effective amount of a type II arabinogalactan of Anoectochilus spp. Also provided are a method for the preparation of the Anoectochilus spp. polysaccharide extract and the use of the extract.

This application claims priority to Taiwan Patent Application No. 098133714 filed on Oct. 5, 2009.

### CROSS-REFERENCES TO RELATED APPLICATIONS

Not applicable.

### BACKGROUND OF THE INVENTION

1. Field of the Invention

The present invention relates to the uses of an Anoectochilus spp. polysaccharide extract for stimulating the growth of advantageous bacteria, stimulating the release of granulocyte colony-stimulating factor (G-CSF), modulating T helper cell type I (Th1 cell), and/or modulating T helper cell type II (Th2 cell), and the preparation method thereof.

2. Descriptions of the Related Art

Anoectochilus spp. belongs to the Orchidaceae family, and it is believed that Anoectochilus formosanus Hayata has broad effects of decreasing blood pressure, reducing blood sugar, protecting the liver, anti-inflammation, anti-cancer, modulating the immune system, etc. Thus, Anoectochilus formosanus Hayata is also called “the king of drugs” or “the tiger of drugs” in Chinese medicines (see US Patent Application Publication No. 2004/0009239A1; Shih et al. 2001. Ameliorative effects of Anoectochilus formosanus extract on osteopenia in overiectomized rats. J Ethnopharmacol 77, 233-238; and Masuda et al. 2008. Suppressive effects of Anoectochilus formosanus extract on osteoclast formation in vitro and bone resorption in vivo. J Bone Miner Metab 26, 123-129, which are entirely incorporated hereinto by reference).

Nevertheless, the active component of Anoectochilus formosanus Hayata remains unclear at present, and research on Anoectochilus formosanus Hayata is restricted to its crude extract, and thus the optimization of drug efficiency and pharmacological study are limited accordingly. Moreover, because the physiological activity of Anoectochilus spp. has not been completely discovered, it is necessary to investigate the application of Anoectochilus spp. to other diseases.

The inventors of the present invention discovered that an Anoectochilus spp. polysaccharide extract has effects of stimulating the growth of advantageous bacteria, stimulating the release of granulocyte colony-stimulating factor (G-CSF), modulating T helper cell type I (Th1 cell), and/or modulating T helper cell type II (Th2 cell) through related in vivo and in vitro experiments, and confirmed that the main active component of the extract is a type II arabinogalactan of Anoectochilus spp.

### SUMMARY OF THE INVENTION

The primary objective of the present invention is to provide an Anoectochilus spp. polysaccharide extract for stimulating the growth of advantageous bacteria, stimulating the release of granulocyte colony-stimulating factor (G-CSF), modulating T helper cell type I (Th1 cell), and/or modulating T helper cell type II (Th2 cell). The extract comprises a type II arabinogalactan of Anoectochilus spp. and has an average molecular weight of about 40 to about 70 kilodaltons.

Another objective of the present invention is to provide a method of the preparation of the aforesaid extract.

Yet a further objective of the present invention is to provide a method for stimulating the growth of advantageous bacteria, stimulating the release of G-CSF, modulating Th1 cell, and/or modulating Th2 cell in a mammal.

The detailed technology and preferred embodiments implemented for the present invention are described in the following paragraphs accompanying the appended drawings for people skilled in this field to well appreciate the features of the claimed invention.

### BRIEF DESCRIPTION OF THE DRAWINGS

FIG. 1 is a flowchart of the preparation of the Anoectochilus spp. polysaccharide extract of the present invention and the type II arabinogalactan of Anoectochilus spp.;

FIG. 2 is a color presenting graph of β-glucosyl-Yariv antigen affinity test of the Anoectochilus spp. polysaccharide extract of the present invention;

FIG. 3A is an analysis graph of monosaccharide composition of the Anoectochilus spp. polysaccharide extract of the present invention;

FIG. 3B is an analysis graph of monosaccharide composition of the type II arabinogalactan of Anoectochilus spp.;

FIG. 4 is an analysis graph of the molecular weight of the Anoectochilus spp. polysaccharide extract of the present invention and the type II arabinogalactan of Anoectochilus spp.;

FIG. 5 is a curve graph of the growth of Bifidobacterium breve;

FIG. 6 is a micro computed tomography graph of femurs of mice.

FIGS. 7A to 7D are bar graphs showing the concentration of various short chain fatty acids in intestines of mice;

FIG. 8A is an electrophoresis graph of mRNA of CaBP-D9k (a calcium-binding protein) in intestines of mice;

FIG. 8B is a bar graph showing the expression of mRNA of CaBP-D9k in intestines of mice;

FIG. 9A is a bar graph showing the nitrite concentration in macrophages RAW 264.7;

FIG. 9B is a bar graph showing the G-CSF concentration in macrophages RAW 264.7

FIG. 9C is a bar graph showing the ratio of G-CSF to nitrogen monoxide in macrophages RAW 264.7;

FIG. 10A is a bar graph showing the TNF-α concentration in the blood of ICR mice after an hour from the administration of the type II arabinogalactan of Anoectochilus spp. to the mice stimulated by lipopolysaccharide (LPS);

FIG. 10B is a bar graph showing the TNF-α concentration in the blood of ICR mice after 16 hours from the administration of the type II arabinogalactan of Anoectochilus spp. to the mice stimulated by LPS;

FIG. 11 is a graph showing the transferring result of the western blotting of T-bet, GATA-3, GAPDH proteins in EL4 cells;

FIG. 12 is a HE stain graph of lung slices of BALB/c mice;

FIG. 13 is a methylene blue stain graph of intestines of BALB/c mice

FIG. 14 is a bar graph showing the turbidity of broth of Bifidobacterium breve; and

FIG. 15 is an electrophoresis graph of mRNAs of nitrogen monoxide synthetase, G-CSF, and TNF-α in macrophages RAW 264.7.

### DESCRIPTION OF THE PREFERRED EMBODIMENT

Arabinogalactans can be classified into type I and type II arabinogalactans, and the main chains of galactan of the type I arabinogalactan are bonded with β(1→4) linkage, whereas the main chains of galactan of the type II arabinogalactan are bonded with β(1→3) (1→6) linkage. Because type II arabinogalactans from different origins have different properties (e.g., molecular weight, structures of main chains or branch chains, components, etc.), their activity is different as well (see Paulsen et al., Bioactive peptic polysaccharides., Adv Polym Sci., 2005, 186: 69-101, which is entirely incorporated hereinto by reference). In this article, “type II arabinogalactan” is defined as “type II arabinogalactan of Anoectochilus spp.”

As described above, the active component of Anoectochilus spp. is still unclear, and Anoectochilus spp. still has many unknown effects. The inventors of the present invention, through many in vitro cell experiments and in vivo animal experiments, discovered that an Anoectochilus spp. polysaccharide extract has new effects of stimulating the growth of advantageous bacteria, stimulating the release of granulocyte colony-stimulating factor (which is called “G-CSF” hereinafter), modulating T helper cell type I (which is called “Th1 cell” hereinafter), and/or modulating T helper cell type II (which is called “Th2 cell” hereinafter), and confirmed that the main active component of the extract is an type II arabinogalactan of Anoectochilus spp.

Thus, the present invention provides an Anoectochilus spp. polysaccharide extract for stimulating the growth of advantageous bacteria, stimulating the release of G-CSF, modulating Th1 cell, and/or modulating Th2 cell. The extract comprises a type II arabinogalactan of Anoectochilus spp.

The Anoectochilus spp. polysaccharide extract of the present invention is a water-soluble extract, and Anoectochilus spp. is preferably Anoectochilus formosanus Hayata. Specifically, the Anoectochilus spp. polysaccharide extract of the present invention mainly comprises polysaccharides, a few proteins, and substantially no fat-soluble components. The proteins present in a form of free or conjugated proteins (such as a glycoprotein or a proteoglycan). After characterization analysis, it was confirmed that the polysaccharide component is mainly comprised of a type II arabinogalactan of Anoectochilus spp. and starch, wherein the starch has a structure of highly branched α(1→4)(1→6) linkage. After the analysis of monosaccharide composition of the Anoectochilus spp. polysaccharide extract and the type II arabinogalactan of Anoectochilus spp., it was discovered that they both comprise arabinose, galactose, glucose, mannose, and fructose. The Anoectochilus spp. polysaccharide extract is mainly comprised of glucose, and the type II arabinogalactan is mainly comprised of galactose.

The Anoectochilus spp. polysaccharide extract of the present invention has an average molecular weight of about 40 to about 70 kilodaltons, and comprises about 20 wt % to about 50 wt % of the type II arabinogalactan (based on the dry weight of the extract). The type II arabinogalactan has an average molecular weight of about 15 to about 45 kilodaltons, and comprises a few proteins presenting in the form of free or conjugated proteins (such as a glycoprotein or a proteoglycan). In a preferred embodiment of the present invention, the Anoectochilus spp. polysaccharide extract has an average molecular weight of about 50 to about 60 kilodaltons, and comprises about 30 wt % to about 40 wt % of the type II arabinogalactan (based on the dry weight of the extract), and the type II arabinogalactan has an average molecular weight of about 25 to about 35 kilodaltons.

The Anoectochilus spp. polysaccharide extract of the present invention has a “prebiotic” effect, and may stimulate the growth of advantageous bacteria (i.e., probiotic) in the intestine. Herein, “advantageous bacteria” refers to bacteria that may carry out physiological reactions beneficial to health or that are capable of curing diseases in animal bodies. In one embodiment of the present invention, Bifidobacterium breve (Bifidobacterium genus) was incubated with the Anoectochilus spp. polysaccharide extract or the type II arabinogalactan of Anoectochilus spp., and the growth of the bacteria was stimulated. In another embodiment of the present invention, it was discovered that the Anoectochilus spp. polysaccharide extract may increase the amount of Bifidobacterium species in mouse intestines.

It is known that the Bifidobacterium species carry out fermentation in the intestine, and may increase the amount of fatty acids, especially short chain fatty acids (such as acetic acid, lactic acid, propanoic acid, and butyric acid), in the intestine. Short chain fatty acids may not only decrease the pH value in the intestine to stimulate calcium absorption, but also activate osteoblasts to promote bone formation to attain the effects of anti-osteoporosis, such as preventing, improving, curing osteoporosis, etc (see Katono et al., sodium butyrate stimulates mineralized nodule formation and osteoprotegerin expression by human osteoblasts. Arch oral Biol. 208; 53:903-909, which is entirely incorporated hereinto by reference). Thus, if the growth of Bifidobacterium species in the intestine can be stimulated, calcium absorption and bone formation may be efficiently promoted to attain the effects of anti-osteoporosis. Because the Anoectochilus spp. polysaccharide extract of the present invention can stimulate the growth of Bifidobacterium species, it can provide the aforesaid anti-osteoporosis effects. In addition, the Anoectochilus spp. polysaccharide extract of the present invention can stimulate the growth of advantageous bacteria in the body, and thus may prevent problems that may arise when using exogenous bacteria to improve osteoporosis; for instance, exogenous advantageous bacteria hardly stay in the intestine for a long time, and the absorption of intestinal mucosa is poor, etc.

The Anoectochilus spp. polysaccharide extract of the present invention also has activity of stimulating macrophages in the body to release G-CSF. In the immune system, leukocytes (white blood cells) play an important role, because when pathogens or exotic substances invade the body, leukocytes may decompose them and induce a series of defensive physiological responses. When a patient is under chemotherapy for cancer, anti-cancer drugs may damage the ability of the patient's body to produce leukocytes, which significantly reduces the amount of leukocytes in the body to make the patient's immunity insufficient and make the patient unable to defend against pathogenic bacteria or viruses. G-CSF is a growth hormone for leukocytes, and may efficiently increase the amount of leukocytes. Because the Anoectochilus spp. polysaccharide extract of the present invention may stimulate macrophages in the body to release G-CSF, it can indirectly increase the amount of leukocytes. Therefore, during the chemotherapy process of a patient with cancer, the patient may be administrated with the Anoectochilus spp. polysaccharide extract of the present invention to improve the side effect of the decrease in leukocytes.

In addition, it is known that G-CSF has effects of anti-inflammation, such as preventing, improving, curing inflammation, etc, and may inhibit the release of TNF-α (tumor necrosis factor-α) stimulated by lipopolysaccharides (LPS) (see Boneberg et al., Molecular aspects of anti-inflammatory action of G-CSF. Inflamm. Res. 2002. 51: 119-128, which is entirely incorporated hereinto by reference). Therefore, the Anoectochilus spp. polysaccharide extract of the present invention may also provide anti-inflammation effects through the stimulation of the release of G-CSF.

The inventors of the present invention also discovered that the Anoectochilus spp. polysaccharide extract of the present invention can modulate Th1 cells and Th2 cells. T cells play a critical role in the immune system, and can differentiate into two kinds of cells depending on the kind of cytokines they secrete. Th1 cells may produce interferon-γ (INF-γ) and interleukin-2 (IL-2), while Th2 cells may produce interleukin-4 (IL-4), interleukin-5 (IL-5), interleukin-6 (IL-6), and interleukin-10 (IL-10). Th1 cells may help killer cells and stimulate cell-mediated immunity by secreting INF-γ to activate macrophages. Th2 cells may assist B cells in producing an atopic antibody, IgE, and may activate mast cells or eosinophils by secreting IL-4 and IL-5 to make them secret inflammatory mediators, including histamine, leukotriene, postaglandine, etc. Th1 cells and Th2 cells may antagonize each other; INF-γ released by Th1 cells may inhibit Th2 cells, and IL-4 and IL-10 released by Th2 cells may inhibit the production of INF-γ from Th1 cells.

Therefore, the interaction between Th1 cells and Th2 cells may influence physiological immune response, and is greatly related to many diseases. For example, it is known that excessive Th2 cell activity may cause allergies, leading to respiratory passage allergies, which causes atopic cough or asthma. In addition, it has been proven in documents that the elevation of immune responses of Th2 cells may stimulate the formation of colon cancer induced by carcinogens (see Osawa et al., Predominant T helper type 2-inflammatory responses promote murine colon cancers. Int J Cancer. 2006. 118(9): 2232-6, which is entirely incorporated hereinto by reference). On the other hand, hyperactive Th1 cell activity may cause abnormality of autoimmune functions. Thus, if the immune balance between Th1 cells and Th2 cells can be modulated to maintain their activity in a normal state, autoimmune diseases can be cured, and allergies (including atopic cough and asthma) can be improved, and colon cancer can be inhibited.

The Anoectochilus spp. polysaccharide extract of the present invention may stimulate the differentiation of Th1 cells and immune responses managed thereby, and may inhibit the differentiation and immune responses of Th2 cells at the same time. Therefore, in the antagonism relationship between Th1 cells and Th2 cells, when the Th2 cell is hyperactive and causing an imbalance between Th1 cells and Th2 cells, the Anoectochilus spp. polysaccharide extract of the present invention may direct the immune responses towards the Th1 cell pathway to modulate the balance between Th1 cells and Th2 cells to attain the anti-allergy effects, improving asthma, inhibiting colon cancer, and modulating immune functions.

The Anoectochilus spp. polysaccharide extract of the present invention can be provided by a method comprising the following steps:
a) extracting Anoectochilus spp. with water to obtain a water-soluble Anoectochilus spp. extract;
b) de-fatting the Anoectochilus spp. extract and then collecting an aqueous extract; and
c) adding ethanol to the aqueous extract and then collecting a precipitate, wherein the amount of ethanol is about 65 vol % to about 85 vol %, based on the total volume of the aqueous extract and ethanol.

An approach for carrying out step a), the extracting step, is described as follows. First, water and Anoectochilus spp. are mixed and agitated to make a juice, and then insoluble components are filtered and removed to obtain a water-soluble Anoectochilus spp. extract. Alternatively, Anoectochilus spp. may be stewed in water, and the stewed solution is collected to obtain the water-soluble Anoectochilus spp. extract.

The de-fatting step b) may be performed with any known suitable de-fatting approaches. For instance, ethyl acetate (or hexane) may be added into the water-soluble Anoectochilus spp. extract to remove fat-soluble components with no desired activity in the Anoectochilus spp. extract, and then an aqueous extract is collected. Herein, there is no limit for the amount of ethyl acetate, and based on the volume of the water-soluble Anoectochilus spp. extract in step a), the amount of ethyl acetate is generally about 15 vol % to about 35 vol %, and preferably is about 20 vol % to about 30 vol % (see Wu et al., The hepatoprotective activity of kinsenoside from Anoectochilus formosanus. Phtother res. 2007; 21: 58-61, which is entirely incorporated hereinto by reference).

In step c), ethanol is added into the aqueous extract, and a precipitate generated is collected. Components in the precipitate are mainly saccharides and a little proteins and nucleic acids. Based on the total volume of the aqueous extract and ethanol, the amount of ethanol is about 65 vol % to about 85 vol %, and preferably is about 70 vol % to about 80 vol %.

Optionally, step a) or b) can be assisted with suitable extracting means (e.g., ultrasonic vibration, etc.) to increase the extracting efficiency. In addition, step a) and/or b) can be optionally repeated to separate effective components from ineffective components in Anoectochilus spp. as much as possible, and extract desired effective components as much as possible so as to decrease the waste of resources and increase economic benefit.

Depending on the application format of the Anoectochilus spp. polysaccharide extract, a drying step d) can be optionally performed to dry the precipitate obtained in step c). For example, if the Anoectochilus spp. polysaccharide extract of the present invention is applied by oral administration, a drying step (e.g., concentrating under a reduced pressure and/or ventilation) can be used to remove organic solvents in the extract to prevent the organic solvents from injuring the body. Alternatively, the precipitate obtained in step c) or d) can be dissolved in water to provide the extract of the present invention as an aqueous solution.

In an embodiment of the present invention, the Anoectochilus spp. polysaccharide extract can be obtained as follows. First, water and Anoectochilus formosanus Hayata are mixed and agitated to make a juice, and insoluble components are filtered and removed to obtain a water-soluble Anoectochilus spp. extract. Then, about 25 vol % ethyl acetate is added into the Anoectochilus spp. extract to perform de-fatting, and the water phase is collected to obtain an aqueous extract. Thereafter, ethanol with a final concentration of about 75 vol % is added into the aqueous extract to generate a precipitate, and then the precipitate is collected to obtain a desired polysaccharide extract of Anoectochilus formosanus Hayata.

The present invention also provides a method for stimulating the growth of advantageous bacteria, stimulating the release of G-CSF, modulating Th1 cells, and/or modulating Th2 cells in a mammal. The method comprises administrating an effective amount of the Anoectochilus spp. polysaccharide extract of the present invention to the mammal. Specifically, the method of the present invention may be used for increasing an amount of fatty acids (e.g., short chain fatty acids) in an intestine, stimulating calcium absorption, anti-osteoporosis, anti-inflammation, inhibiting a decrease of leukocytes, anti-allergy, improving asthma, inhibiting colon cancer, and/or modulating immunological functions, etc.

The Anoectochilus spp. polysaccharide extract of the present invention can be administrated as a medicament in any suitable format for different way. For example, but not limited thereby, the medicament can be applied by oral administration, subcutaneous administration, or intravenous administration, etc. In addition to the extract of the present invention, the medicament may contain one or more adjuvants and can be used in both veterinary medicine and human medicine in practice.

In terms of the manufacture of a medicament suitable for oral administration, the extract of the present invention can optionally be mixed with adjuvants that are suitable for oral administration and do not influence the activity of the extract of the present invention adversely. For example, the adjuvants can be a solvent, an oil solvent, a thinner, a stabilizer, an absorption-retarding reagent, a disintegrant, an emulsifier, a binder, a lubricant, a deliquescent, etc. For instance, the solvent can be water or a sucrose solution; the thinner can be lactose, starch, or microcrystalline cellulose; the absorption-retarding reagent can be chitosan or glycosaminoglycan; the lubricant can be magnesium carbonate; and the oil solvent can be plant oil or animal oil, such as olive oil, heliotrope oil, fish liver oil, etc. Herein, with a conventional method, the extract of the present invention and other suitable adjuvant(s) can be made into a suitable oral administration form, such as a tablet, a capsule, a granule, a powder, a fluid extract, a solution, a syrup, a suspension, an emulsion, a tincture, etc.

As for the manufacture of a medicament suitable for a subcutaneous or intravenous administration, the extract of the present invention and optional adjuvant(s) can be mixed with one or more components conventionally used for these forms (e.g., a hydrotropic agent, an emulsifier, or other adjuvants), to produce an intravenous injection, an emulsion intravenous injection, an injection, a powder injection, a suspension injection, a powder-suspension injection, etc. For instance, the solvent can be water, a physiological solution of sodium chloride, alcohols (e.g., ethanol, propanol, glycerin, etc), a sugar solution (e.g., a glucose or mannitol solution), or combinations thereof.

Optionally, in addition to the above useful adjuvants, other additives, such as a flavoring agent, a toner, a coloring agent, etc, can also be added during the manufacture of the medicament to enhance the sense of comfort for the mouth and visual feelings during the administration. A suitable dosage of a preservative, a conservative, an antiseptic, an anti-fungus reagent, etc, also can be added to improve the storability of the resulting medicament.

Furthermore, the medicament may optionally contain one or more other active components to enhance its effect or increase its flexibility in application and formulation. For example, other active components that can be included in the medicament comprise substances for treating osteoporosis (e.g., alendronate, parathorine, estrogen, calcium compounds, or Vitamin D, etc), anti-arthritis substances (e.g., chondroitin or glucosamine), other active components, etc, as long as the other active components have no adverse effects on the extract of the present invention.

Depending on the requirements of a subject, the medicament can be applied with different administration frequency, such as once a day, several times a day, or once for several days, etc. For instance, when the medicament is used for anti-inflammation in a mammal, the amount of the Anoectochilus spp. polysaccharide extract administrated to the mammal, calculated as the type II arabinogalactan, is about 2 mg/kg-body weight to about 25 mg/kg-body weight per day, wherein the unit “mg/kg-body weight” means the dosage required for per kilogram body weight. Preferably, the amount of the Anoectochilus spp. polysaccharide extract administrated to the mammal, calculated as the type II arabinogalactan, is about 3 mg/kg-body weight to about 20 mg/kg-body weight per day. However, in acute situations (e.g., acute arthritis or serious osteoporosis), the dosage can be increased to several times or several tens of times, depending on the practical requirements.

The present invention will be further illustrated in details with specific examples as follows. After referring to the examples described in the following paragraphs, people skilled in this field can easily appreciate the basic spirit and other invention purposes of the present invention, and technical methods adopted in the present invention and better embodiments. However, the following examples are provided only for illustrating the present invention, and the scope of the present invention is not limited thereby.

### Example 1

### Preparation of a Water-Soluble Polysaccharide Extract of Anoectochilus formosanus Hayata

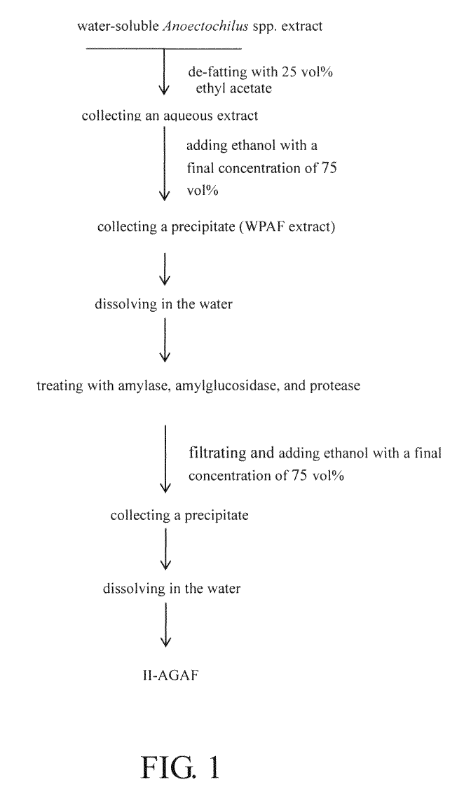

As illustrated below, according to the flow chart as shown in FIG. 1, an extract of water-soluble polysaccharide of Anoectochilus formosanus Hayata (which is called “WPAF extract” hereinafter) and a type II arabinogalactan of Anoectochilus formosanus Hayata (which is called “II-AGAF” hereinafter) were prepared.

First, water and Anoectochilus formosanus Hayata (a sample of this plant has been deposited in the Pharmacy College, China Medical University, Taiwan, with a deposition number of CMU AF 0609, and has been identified by the college) purchased from Yu-Jung farm (Puli, Taiwan) were mixed and made into a juice, and insoluble components were filtered and removed to obtain a water-soluble Anoectochilus formosanus Hayata extract. Then, ethyl acetate with a final concentration of about 25 vol % was added into the Anoectochilus formosanus Hayata extract, and an aqueous extract was collected to remove fat-soluble components. Ethanol with a final concentration of about 75 vol % was then added into the aqueous extract to generate a precipitate, and the precipitate was collected and dissolved in the water to obtain an extract of water-soluble polysaccharide of Anoectochilus formosanus Hayata (a WPAF extract), and the yield of which is about 2.4 milligram per kilogram of Anoectochilus formosanus Hayata.

The water-soluble WPAF extract was divided into two portions, and in one portion, amylase, amylglucosidase, and protease (purchased from Megazyme International, Wicklow, Ireland) were added thereinto to perform degradation, and ethanol with a final concentration of about 75 vol % was added to generate a precipitate. Finally, the precipitate was dissolved in the water to obtain a type II arabinogalactan (II-AGAF), which is a polysaccharide that was not completely purified.

### Example 2

### Characterization of the WPAF Extract and II-AGAF

I. Color Presenting Test Using Iodide

The principle of color presenting using iodide is that polysaccharides (e.g., starch or glycogen (both are glucan)) may form into complexes with iodide and generate color, in which iodide may locate in the middle of a spiral molecule chain of polysaccharides, and color generated depends on the length of a straight chain (α(1→4) linkage). For instance, amylose may generate purple; dextrin, a partial hydrolyzed product from starch, may generate a reddish brown to transparent color depending on the chain length, and glycogen with branched chains may generate reddish brown.

The WPAF extract from Example 1 (12 mg) was added into 90% dimethyl sulfoxide (6 ml) and heated under 100° C. for 30 minutes. Then, 100 μl of the obtained sample was mixed with 900 μl water, and 50 μl of 0.01 N iodide-potassium iodide was added thereinto, and the mixture was vibrated and mixed.

The result of the color presenting test using iodide showed that the WPAF extract may generate red to purple, which indicates that α-dextrorotary glucan in the WPAF extract has highly branched structure with α(1→4)(1→6) linkage.

II. β-glucosyl-Yariv Antigen Affinity Test

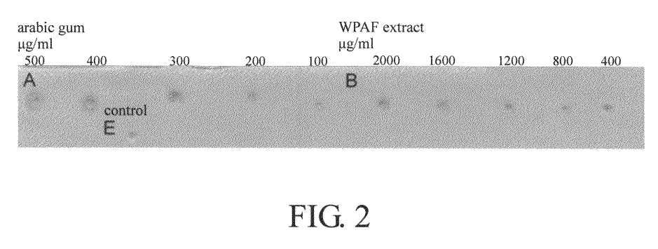

Yariv test was carried out according to van Holst's and van Hengel's methods (see van Hoist et al., Quantification of arabinogalactan protein by single radical gel diffusion. Anal Biochem. 148:446-450, 1985 and van Hengel et al., Fucosylated arabinogalactan-proteins are required for full root cell elongation in Arabidopsis. Plant J. 32:106-113, 2002, which are entirely incorporated hereinto by reference), and Arabic gum was used as a positive control. In deionized water, a gel piece was heated and dissolved with 1 wt % Agarose I™, 0.15 M sodium chloride, 0.02 w/v % sodium azide, and 10 μg/ml β-glucosyl-Yariv antigen, and was instilled into a gel casting cassette (Protean II, BioRad, Hercules, Calif., USA) to make a gel piece with a thickness of 3 mm. Then, a sample loading hole with a diameter of 1.2 mm was made with a Pasteur pipet (Kimble, Toledo, Ohio, USA). After the WPAF extract from Example 1 was dissolved with 0.15 M sodium chloride and a 0.02 w/v % sodium azide solution, 0.8 μl of the extract was loaded into the sample loading hole. The gel piece was placed in a humid closed container under room temperature for two days, and a diffusion circle was observed. The results are shown in FIG. 2.

Arabinogalactan can be classified into type I and type II, and the main chains of galactan of the type I arabinogalactan are bonded with β(1→4) linkage, whereas the main chains of galactan of the type II arabinogalactan are bonded with β(1→3)(1→6) linkage. The Yariv reagent, a synthesized phenylglycoside, may react with the type II arabinogalactan to generate color, so it can be used to determine the linkage form of arabinogalactan.

FIG. 2 shows that the Yariv reaction had occurred and color was generated as the WPAF extract reacted with Arabic gum, indicating that the WPAF extract contains the type II arabinogalactan.

The above results indicate that the WPAF extract mainly comprises starch and II-AGAF. Thus, if the WPAF extract is treated with amylase and amylglucosidase, starch can be degraded to leave a major residue as II-AGAF. In addition, since polysaccharide components generally contain proteins (e.g., glycoproteins), the components may be treated with proteases to further remove proteins.

III. Determination of the Content of Proteins, Saccharides, and Uronic Acids

The total content of proteins was measured with the Folin-Lowry method and the Coomassie Blue method, and bovine serum albumin was used as a standard (see Lowry et al., Protein measurement with the Folin phenol reagent. J. Biol. Chem. 1951. 93: 265-275, which is entirely incorporated hereinto by reference). The results are shown in Table 1.

The total content of saccharides was measured with the phenol-sulfuric acid method, and glucose was used as a standard for the WPAF extract, and galactose was used as a standard for II-AGAF (see Dubois et al., Colorimetric method for determination of sugars and related substances. Anal. Chem. 1956. 28: 350-356, which is entirely incorporated hereinto by reference). The results are shown in Table 1.

The content of uronic acids was measured by using m-hydroxydiphenyl, in which a standard curve was made by using 10 to 90 μg/ml galacturonic acid, and a solution of 0.5 wt % sodium hydroxide was used to replace a m-hydroxydiphenyl solution to correct a brown-presenting reaction of neutral saccharides.

First, 200 μg of the WPAF extract was added into a sulfuric acid solution (1.2 ml) containing 0.0125 M sodium tetraborate, and was mixed thoroughly and bathed in boiling water for five minutes. After the sample was cooled, a m-hydroxydiphenyl solution (20 μl) or a solution of 0.5 wt % sodium hydroxide was added thereinto, and was placed still for 15 minutes to generate color. Absorbance values were detected with a wavelength of 520 nm, and the results are shown in Table 1. The aforesaid method can be seen in Blumenkrantz et al., New method for quantitative determination of uronic acid. Analytical Biochemistry. 1973. 54: 484-489, which is entirely incorporated hereinto by reference.

TABLE 1

| | | Composition (%) | | | |
|---|---|---|---|---|---|
| | Yield^{a} | Proteins | Proteins | | Uronic |
| | (%) | (FL^{b}) | (CB^{c}) | Carbohydrates^{d} | acid^{e} |
| WPAF extract | 100 | 7.3 | 0.6 | 24.6 | 2.0 |
| II-AGAF | 33.4 | 3.5 | 0.2 | 19.1 | 2.3 |

^{a}Using the solid content of the WPAF extract as basis.
^{b}Values from Folin-Lowry (FL) method using BSA as standard
^{c}Values from Coomassie Blue (CB) method (Bio-Rad protein assay reagent) using BSA as standard.
^{d}Values from the phenol-sulfuric acid method using glucose or galactose as standard for the WPAF extract or II-AGAF
^{e}Using galacturonic acid as standard

As shown in Table 1, the WPAF extract contains about 33.4 wt % of II-AGAF, and II-AGAF contains some proteins.

IV. Analysis of Monosaccharide Ratio

After polysaccharides in the WPAF extract were hydrolyzed into monosaccharides with an acid, HPAEC (high performance anion-exchange chromatograph) was used to analyze the monosaccharide ratio of the WPAF extract to determine the monosaccharide composition in the WPAF extract and II-AGAF from Example 1, in which the hydrolysis was performed mainly with 2 M trifluoroacetic acid.

First, the WPAF extract (1 mg) or II-AGAF (1 mg) was dissolved in a solution of 2M trifluoroacetic acid (1 ml), and was placed in a hydrolysis tube under a vacuum condition to carry out acid hydrolysis, and the reaction condition was 100° C. for three hours. After the reaction was completed, trifluoroacetic acid was removed by concentrating under a reduced pressure, and deionized water was added into the sample, and the sample was dried by concentrating under a reduced pressure again. The aforesaid procedure can be repeated for several times to possibly remove trifluoroacetic acid.

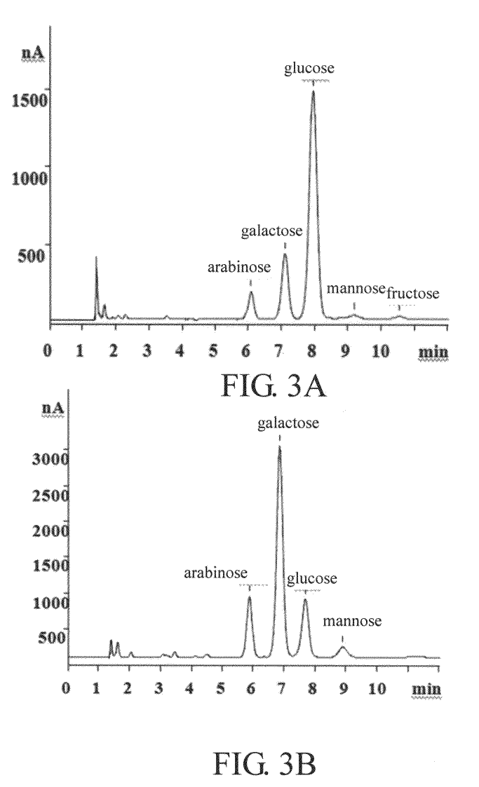

Then, HPAEC analysis was conducted. The above hydrolyte was dissolved with 1 ml deionized water, and was filtered by a PVDF membrane with a pore size of 0.45 μm (Millipore, Milford, Mass., USA), and was injected into a HPAEC system to conduct the analysis. The elements and analysis condition of the HPAEC system were as follows. 817 Bi Bioscan (Metrohm, Herisau, Swiss); 812 valve unit (Metrohm, Herisau, Swiss); Series III pump (LabAlliance, Pennsylvania, USA); detector: pulsed amperometric detector, Metrohm, Herisau, Swiss); potential and pulse time: E1: 0.05 volt/0.4 s, E2: 0.75 volt/0.2 s, E3: −0.15 volt/0.4 s; sample loop: capacity: 20 μl; separation column: CarboPac PA1 guard column (4×50 mm)—CarboPac PA1 (4×250 mm, Dionex, Sunnyvale, Calif., USA); flow rate: 1.0 ml/min; elution system; 10 mM sodium hydroxide and 1 mM barium acetate (filtered by a Nylon membrane of 0.2 μm pore size (ChromTech, Apple Valley, Minn., USA); Data processing system; Metrodata IC Net 2.1 (Metrohm, Herisau, Swiss). Arabinose, galactose, glucose, mannose, and fructose were used as standards, and the analysis was conducted by comparing retention time. The results are shown in Table 2, FIGS. 3A and 3B.

TABLE 2

| | Molar percentage (%) | | | | |
|---|---|---|---|---|---|
| | Arabinose | Galactose | Glucose | Mannose | Fructose |
| WPAF extract | 12.76 | 17.56 | 61.30 | 5.70 | 2.68 |
| II-AGAF | 22.38 | 56.54 | 15.35 | 5.73 | trace |

As shown in FIG. 3A, according to the HPAEC analysis, the WPAF extract comprises monosaccharide units like glucose, galactose, arabinose, mannose, and fructose, the ratios of which are shown in Table 2. Glucose had the highest ratio.

As shown in FIG. 3B, II-AGAF obtained after the degradation of the WPAF extract with amylase and amylglucosidase still contains monosaccharide units like glucose, galactose, arabinose, mannose, and fructose, the ratios of which are shown in Table 2. Galactose had the highest ratio.

The results indicate that the WPAF extract of the present invention mainly comprises starch and arabinogalactan with galactan as the backbone. The side chains of the arabinogalactan contain glucose and mannose units.

V. Determination of Molecular Weight

The molecular weight of the WPAF extract and II-AGAF in Example 1 was analyzed with HPSEC (high performance size exclusion chromatograph). A suitable amount of the WPAF extract or II-AGAF was dissolved with deionized water, and was filtered by a PVDF membrane with a pore size of 0.45 μm (Millipore, Milford, Mass., USA), and was injected into a sample loop with a capacity of 500 μl, and was analyzed with a HPSEC instrument, in which standards for the molecular weight analysis is STANDARD P-82 (pullulans: P-800 (78.8×10⁴ Dalton), 400 (40.4×10⁴ Dalton), 200 (21.2×10⁴ Dalton), 100 (11.2×10⁴ Dalton), 50 (4.73×10⁴ Dalton), 20 (2.28×10⁴ Dalton), 10 (1.18×10⁴ Dalton), and 5 (0.59×10⁴ Dalton), Shodex, Tokyo, Japan).

The condition of the HPSEC analysis is as follows. Washing solution feeding pump: 709 IC pump (Metrohm, Switzerland); injector: Rheodyne sample injector (Cotati, Penn., USA); sample injection volume: 500 μL; constant-temperature cabinet for a column: Super CO-150 (Enshine, Taiwan), maintained at 70° C.; Detector: Multi Angle Laser Light Scattering photometer (DAWN EOS, Wyatt Technology Inc., Santa Barbara, USA); Interferometric Refractometer (OPTILAB DSP, Wyatt Technology Inc., Santa Barbaraa, USA), connected in series, the temperature of refractometer being maintained at 35° C.; column: Tskgel guard column PWH (75×7.5 mm i.d., Tosho, Tokyo, Japan)+TSKgel G4000 PWXL (300×7.8 mm i.d., Tosho, Tokyo, Japan)+ViscoGel G2500 PWXL (300×7.8 mm i.d., Viscotek, Tex., USA); flow phase: 0.3 N sodium nitrite (NaNO₃)+0.02% sodium azide (NaN₃); flow rate: 0.8 ml/min.

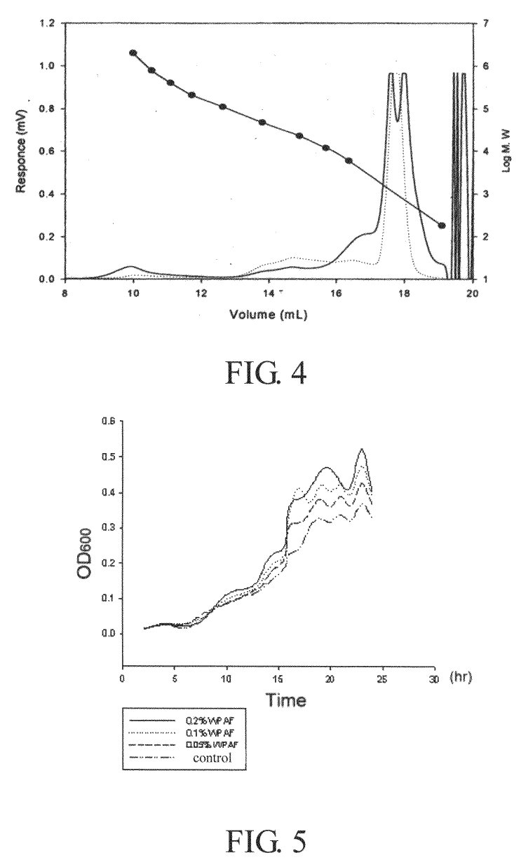

The analysis result is shown in FIG. 4. In FIG. 4, the solid line represents the WPAF extract, and the dashed line represents II-AGAF. After calculation, the average molecular weight (M_{w}) of the WPAF extract is 55 kDal, and that of II-AGAF is 29 kDal.

### Example 3

### Prebiotic Effect of the WPAF Extract

I. Tube Test

Bifidobacterium breve was purchased from Food Industry Research and Development Institute, Hsin Chu, Taiwan, and was incubated in a MRS medium (comprising 1 wt % proteose No. 3, 1 wt % beef extract, 0.5 wt % yeast extract, 2 wt % dextrose, 0.1 wt % Tween 80, 0.2 wt % ammonium citrate, 0.5 wt % sodium acetate, 0.01 wt % magnesium sulfate, 0.005 wt % manganese, 0.2 wt % potassium hydrogen phosphate, and 1.5 wt % agar (Difco, Md., USA)). After the bacteria were incubated in an incubator under an anaerobic condition for one or two days, the broth was collected, and turbidity was measured with a wavelength of 600 nm. Then, the broth was appropriately diluted, and was uniformly smeared on the surface of a MRS medium in a plate, and was incubated in an incubator at 37° C. under an anaerobic condition. After two days, the turbidity of the medium was measured to calculate the number of colonies.

Turbidity is linearly proportional to the number of the colonies calculated after the incubation by smearing the broth on the MRS medium, and a formula and r value representing their relationship are as follows:

Y=(1361X−11.74)×10⁷, r²=0.9871, Y=colony forming unit (CFU)/ml, X=OD600

The above formula indicates that turbidity may reflect the number of colonies, and thus the number of colonies in the following experiments is represented by turbidity.

As shown in FIG. 5, in the curve graph of the incubation time of Bifidobacterium breve versus turbidity, a maximum slope presents at 18 hours after the incubation, which is 0.05. If the WPAF extract in Example 1 with various concentrations was added into a medium containing Bifidobacterium breve, the maximum slope of the growth curve of Bifidobacterium breve was shifted from 18 hours to 16 hours. The slopes at 16 hours of the WPAF extract with various concentrations of 0.5, 1, and 2 mg/ml are 0.08, 0.10, and 0.10, respectively, and the slope at 18 hours of the control group is 0.06. This result indicates that the WPAF extract of the present invention may promote the growth of Bifidobacterium breve. In the control and experiment groups, the turbidity of Bifidobacterium breve incubated after 18 hours is shown in Table 3.

TABLE 3

| | Concentration | OD |
|---|---|---|
| | (mg/ml) | B. breve |
| Control | 0 | 0.188 ± 0.004 |
| WPAF extract | 0.5 | 0.304 ± 0.003** |
| WPAF extract | 1.0 | 0.312 ± 0.005*** |
| WPAF extract | 2.0 | 0.321 ± 0.005*** |

All values are mean ± SD (n = 6).
**P < 0.01,
***P < 0.001, compared with the control group.

II. Mouse Intestine Test

ICR mice (Purchased from BioLASCO, Taipei, Taiwan) were used in the following experiments. First, the water or the WPAF extract in Example 1 (15 or 40 mg/kg-body weight) was administrated to mice, and after three and seven days, mouse stool was collected with a sealed container. The stool was then diluted with a sterile anaerobic diluent in a proper proportion, and was mixed uniformly with a tube vibrator to form a homogeneous—liquid. Under an anaerobic condition, the liquid was diluted in a proper concentration, and was smeared on the surface of a Beerens medium (1 L medium comprises 37 g brain heart infusion, 5 g yeast extract, 0.5 g cysteine, and 15 g agar) containing Bifidobacterium, which was then incubated in an incubator at 37° C. under an anaerobic condition for two to four days, and the number of colonies was calculated.

The results are shown in Table 4. At the third day, the WPAF extract with dosage of 45 mg/kg-body weight obviously increased the number of colonies of Bifidobacterium in mouse stool, and at the seventh day, the WPAF extract with dosage of 15 and 45 mg/kg-body weight both obviously increased the number of colonies of Bifidobacterium in the stool.

TABLE 4

| | |
|---|---|
| Dosage | Log10 CFU/g stool |

| | (mg/kg) | Day 3 | Day 7 |
|---|---|---|---|
| H₂O | | 6.2 ± 0.5 | 6.2 ± 0.5 |
| WPAF extract | 15 | 6.8 ± 0.7 | 7.2 ± 0.1*** |
| WPAF extract | 45 | 7.5 ± 0.2*** | 7.5 ± 0.2*** |

All values are mean ± SD (n = 7).
***P < 0.001, compared with the H₂O group.

### Example 4

### Anti-Osteoporosis Effect of the WPAF Extract

I. Ovariectomized Mice Test

ICR mice (Purchased from BioLASCO Biotech, Taiwan) were used in the following experiments. It is known that insufficient secretion of estrogen may lead to osteoporosis, and thus, in this experiment, ovaries of the ICR mice were removed to make them unable to secret estrogen to induce osteoporosis. In the control group (i.e., a sham group), the skin and muscle on the ovary positions at both sides of the dorsum of the ICR mice were incised and then sutured without removing the ovaries.

After three days, various dosage (15 or 45 mg/kg-body weight) of the WPAF extract in Example 1 was administrated to the ICR mice. After three weeks from the administration, the mice were sacrificed. Hereinafter, the mice of which ovaries were removed are called “OVX” or “OVX mice.”

The amount of C-terminal cross-linked telopeptides of type I collagen (CTx) in the serum of the mice was determined by ELISA (enzyme linked immunosorbent assay), and the experiment agents were purchased from IDS Nordic A/S, Herlev, Denmark. CTx is a degraded product of collagen in bones, and if the CTx concentration in the blood raises, it means that bone resorption increases, which may cause osteoporosis (see Swaminathan. 2001. Biochemical markers of bone turnover. Clinica Chimica Acta. 313: 95-105, which is entirely incorporated hereinto by reference). The results are shown in Table 5.

TABLE 5

| | Dose | |
|---|---|---|
| | (mg/kg-body | Serum CTx |
| | weight) | (ng/ml) |
| Control | 0 | 24.4 ± 3.4 |
| OVX + H₂O | 0 | 36.9 ± 5.2^{##} |
| OVX + WPAF extract | 15 | 30.2 ± 6.2 |
| OVX + WPAF extract | 45 | 27.6 ± 4.8* |

All values are mean ± SD (n = 8).
^{##}P < 0.01, compared with the H₂O group.
*P < 0.05 compared with the OVX + H₂O group.

As shown in Table 5, removing ovaries may increase the CTx concentration in serum of the mice, and the WPAF extract of the present invention may lower the CTx concentration, indicating that it can inhibit bone resorption to reduce the loss of bone matrix.

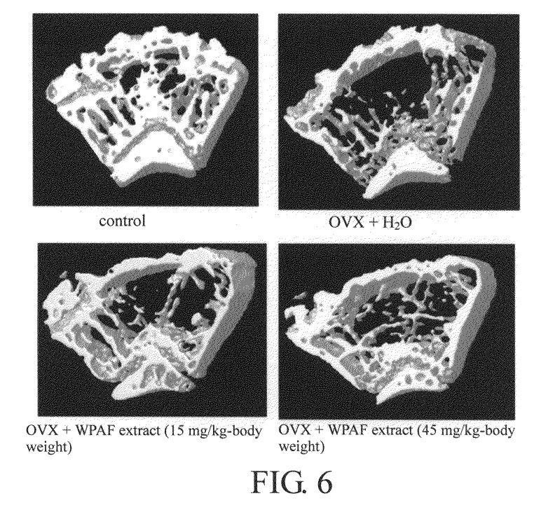

Thereafter, femurs of the mice were taken out, and were pictured with a micro computed tomography instrument (SkyScan 1076, Kontizh, Belgium). The trabecular number and the ratio of bone volume (BV) to tissue volume (TV) were analyzed with an analysis software. The results are shown in FIG. 6 and Table 6.

TABLE 6

| | Dosage | | |
|---|---|---|---|
| | (mg/kg-body | BV/TV | trabecular number |
| | weight) | (%) | (/mm) |
| Control | 0 | 27.6 ± 2.6 | 12.4 ± 2.2 |
| OVX + H₂O | 0 | 16.7 ± 1.6^{###} | 9.0 ± 1.8^{##} |
| OVX + WPAF extract | 15 | 19.6 ± 0.9 | 10.4 ± 1.5 |
| OVX + WPAF extract | 45 | 20.2 ± 2.7* | 12.2 ± 1.3* |

All values are mean ± SD (n = 8).
^{##}P < 0.01, compared with the control group.
*P < 0.05, compared with the OVX + H₂O group

As can be seen in FIG. 6 and Table 6, the WPAF extract of the present invention may inhibit the reduction of the bone volume ratio and the reduction of the trabecular number in the OVX mice.

Thereafter, vertebrae of the mice were taken out, and flesh thereon was removed thoroughly, and the vertebrae were bathed in ethanol to remove fat, and were dried at 100° C. overnight, and the weight of which was measured. The vertebrae were then burned at 1000° C. for ten hours, and the ash weight was measured. The ash was dissolved in 6N hydrochloric acid. The amount of calcium in the ash was determined using o-cresolphthalein complexone, and the calcium weight of the vertebrae per gram was calculated. Assay agents were purchased from Randox Lab. Ltd., England. The results are shown in Table 7.

TABLE 7

| | Dosage | |
|---|---|---|
| | (mg/kg-body | |
| | weight) | Ca/Bone weight (%) |
| Control | 0 | 14.8 ± 0.8 |
| OVX + H₂O | 0 | 12.3 ± 0.6^{###} |
| OVX + WPAF extract | 15 | 13.1 ± 0.1 |
| OVX + WPAF extract | 45 | 14.1 ± 0.4** |

All values are mean ± SD (n = 8).
^{###}P < 0.001, compared with control group.
**P < 0.01, compared with the OVX + H₂O group.

As indicated in Table 7, the WPAF extract of the present invention may inhibit the loss of calcium in the vertebrae of the OVX mice. From FIG. 6 and Tables 5 to 7, the above experiments show that the WPAF extract of the present invention can improve osteoporosis of the mice caused by removing their ovaries.

II. Calcium Absorption Test

Substances in the cecum of the above sacrificed ICR mice were taken out and centrifuged, and the supernatant was collected. After the supernatant was diluted properly, calcium concentration therein was determined with o-cresolphthalein complexone. The results are shown in Table 8.

TABLE 8

| | Dosage | |
|---|---|---|
| | (mg/kg-body | Cecum Ca |
| | weight) | (mg/dl) |
| Control | 0 | 18.2 ± 4.0 |
| OVX + H₂O | 0 | 17.1 ± 2.0 |
| OVX + WPAF extract | 15 | 23.1 ± 5.8* |
| OVX + WPAF extract | 45 | 24.4 ± 3.1* |

All values are mean ± SD (n = 8).
*P < 0.05, compared with the OVX + H₂O group.

As shown in Table 8, the WPAF extract of the present invention may increase the amount of free calcium in the cecum of the mice. Because the acidic environment may elevate calcium solubility, it makes calcium easy to be absorbed by the intestine. Thus, the following experiments further determine the amount of short chain fatty acids of the substances in the cecum of the mice.

The above supernatant was analyzed with an HPLC instrument. The elements of the HPLC instrument are as follows. Pump Delivering system: SDS 9414 Solvent Delivery System (Schambeck SFD GmbH, Bad Honnef, Germany); sample injection capacity: 20 μL; Detector: 115 UV Detector (Gilson, Wis., USA) at 210 nm, and refractometer: Shodex RI-71 (SHOWA DENKO, Tokyo, Japan); column: Transgenomic ICSep ION-300 column (Transgenomic, CA, USA) and guard column: Transgenomic ICSep ION-300 Guard kit (Transgenomic, CA, USA); the temperature of an oven for column: 65° C.; eluent: 0.0085N H₂SO₄; data processing system: SISC Hsun-Hua chromatography integration data processing system (Hsun-Hua, Taipei, Taiwan).

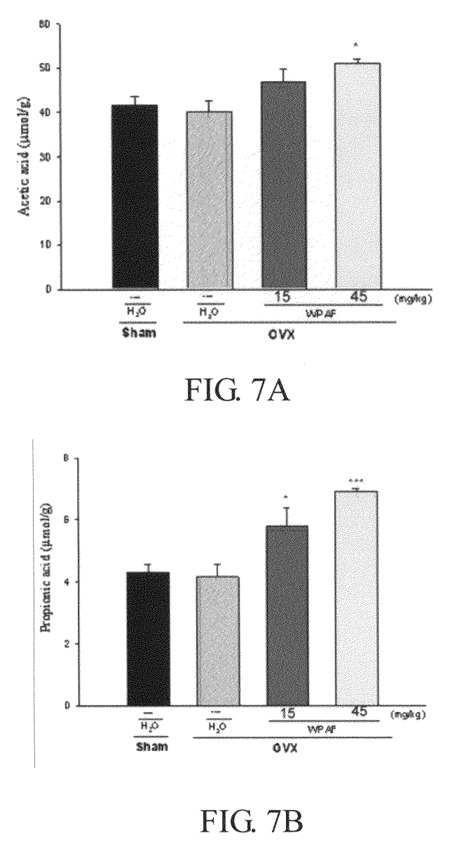

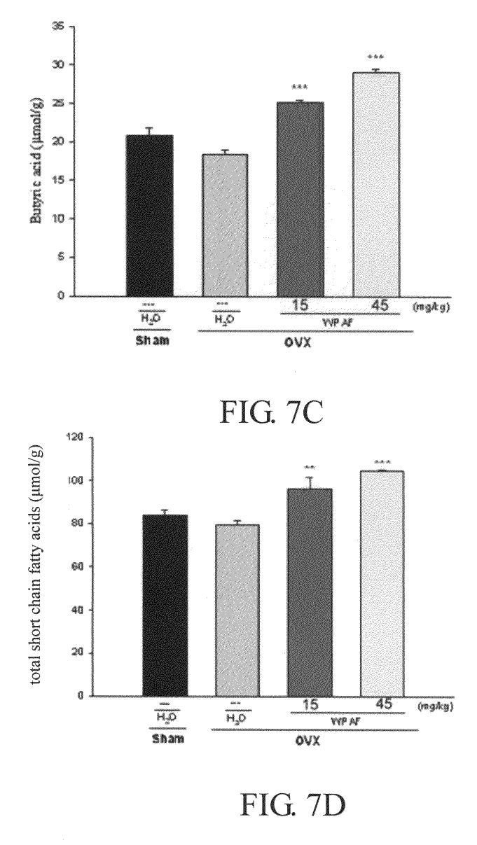

The above supernatant was injected into the system, and absorbance values were determined with an ultraviolet detector at a wavelength of 210 nm. Because the wavelength of the largest absorbance value of organic acids is 210 nm, the wavelength can be used to detect short chain fatty acids, and the concentration thereof can be measured with a refractometer. In this experiment, short chain fatty acid standards (including acetic acid, lactic acid, propanoic acid, and butyric acid) with different concentrations were prepared, and a standard curve was made according to the integration area of peaks in analysis diagrams and concentrations to calculate the concentration of short chain fatty acids in the supernatant. A unit “μmole/g-substances in cecum” is used to represent the amount of acetic acid, propanoic acid, and butyric acid. The results are shown in FIGS. 7A to 7D.

As can be seen in FIGS. 7A to 7D, the WPAF extract of the present invention can increase the amount of short chain fatty acids in the cecum of the mice, and thus may decrease the pH value in the cecum to promote calcium absorption.

CaBP-D9k (a calcium-binding protein) of intestinal mucosa cells is closely related to calcium absorption, and as the expression amount of CaBP-D9k increases, calcium adsorption increases as well (see Bouillon et al., 2003. Intestinal calcium absorption: molecular vitamin D mediated mechanisms. J. Cell. Biochem., 88: 332-339, which is entirely incorporated hereinto by reference). Therefore, the following experiment further analyzed the mRNA amount of CaBP-D9k to observe the expression amount thereof.

First, the cecum mucosa of the ICR mice was scraped and collected, and mRNAs therein were extracted. The mRNA expression amount of CaBP-D9K in the cecum and colon mucosa was analyzed by RT-PCR. The primer sequences for analyzing CaBP-D9K are as follows:

| | | |
|---|---|---|
| sense: | AAGAGCATTTTTCAAAAATA | (SEQ ID NO: 1) |
| anti-sense: | GTCTCAGAATTTGCTTTATT | (SEQ ID NO: 2) |

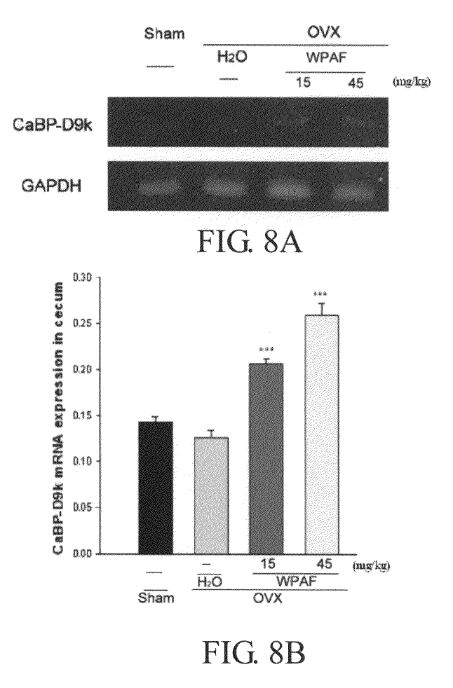

As can be seen in FIGS. 8A and 8B, the WPAF extract promotes the expression of mRNA of CaBP-D9K in the cecum, indicating that the WPAF extract can stimulate calcium absorption.

Examples 3 and 4 indicate that the WPAF extract of the present invention may elevate the amount of advantageous bacteria in the intestine by stimulating the growth of the advantageous bacteria to enhance the fermentation in the intestine, which may generate short chain fatty acids to lower the pH value to promote calcium absorption. Thus, the WPAF extract of the present invention can improve osteoporosis.

### Example 5

### The WPAF Extract Stimulates Macrophages to Release G-CSF

I. Macrophage Test in Mice

First, 5 wt % thioglycollate (Becton Dickinson, Franklin Lakes, N.Y., USA) was administrated to ICR mice by peritoneal cavity injection. After three days, macrophages in the peritoneal cavities of the ICR mice were washed out with Hank's Balanced Salt Solutions (Amresco, Solon, Ohio, USA), and were incubated in a medium (comprising Dulbecco's modified eagle's medium, 10% heat-inactivated fetal bovine serum, 100 U/ml penicillin, and 100 μg/ml streptomycin). Then, the WPAF extract in Example 1 (50, 100, or 200 μg/ml) was added into the medium containing macrophages, which were kept being incubated. After 16 hours, the broth was centrifuged, and the supernatant was collected, and the G-CSF concentration in serum of the mice was determined by ELISA. The results are shown in Table 9.

TABLE 9

| | G-CSF (pg/ml) |
|---|---|
| Control | 0.93 ± 1.98 |
| WPAF extract (50 μg/ml) | 909.09 ± 176.82* |
| WPAF extract (100 μg/ml) | 2019.52 ± 821.10** |
| WPAF extract (200 μg/ml) | 3139.54 ± 332.02** |

All values are mean ± SD (n = 3).
*P < 0.05,
**P < 0.01, compared with the control group.

As can be seen in Table 9, the WPAF extract of the present invention obviously activates macrophages in the peritoneal cavities of the mice to make them release G-CSF.

II. Leukocyte Test

Cyclophosphamide is a drug for chemotherapy, which has immunity inhibition effect and may reduce the amount of leukocytes (white blood cells). First, the WPAF extract in Example 1 (15 or 45 mg/kg-body weight) was administrated to ICR mice through peritoneal cavity injection continuously for three days. At the third day, after 30 minutes from the administration, 100 mg/kg-body weight of cyclophosphamide was injected into peritoneal cavities of the mice. At the second day after the administration of cyclophosphamide, the blood of the mice was collected from orbits, and micronuclei therein were determined. A proto type MicroFlow analysis kit (comprising FITC-anti CD71 and propidium iodide) for mouse micronuclei was used to conduct the determination, and a flow cytometry (Becton Dickinson FASCScan) was used to count 600 to 950 polychromatic erythrocytes (reticulocytes), the number of micronuclei, and the ratio of polychromatic erythrocytes to all red blood cells. The above experiment procedure can be seen in Hayashi et al., 1990. The micronucleus assay with mouse peripheral blood reticulocytes using acridine orange-coated slides. Mutat. Res. 245: 245-249, which is entirely incorporated hereinto by reference.

During the experiment, the WPAF extract in Example 1 was administrated to ICR mice everyday, and the ICR mice were sacrificed at the seventh day after the administration of cyclophosphamide. Spleens of the mice were taken out and the weight thereof was measured, and the change of leukocytes was analyzed with a blood cell analysis instrument, and the G-CSF concentration in serum was also analyzed. The results are shown in Tables 10 and 11.

TABLE 10

| | Dosage | No. | | |
|---|---|---|---|---|
| | (mg/kg-body | of | PCE/NCE | % of MN |
| | weight) | PCE | (%) | in PCE |
| Control | | 927 | 1.98 ± 1.99 | 3.58 ± 3.52 |
| CP + H₂O | | 625 | 0.37 ± 0.30^{###} | 16.1 ± 12.4^{#} |
| CP + WPAF extract | 15 | 693 | 0.63 ± 0.71 | 12.6 ± 12.2 |
| CP + WPAF extract | 45 | 617 | 0.43 ± 0.31 | 14.8 ± 15.6 |

All values are mean ± SD (n = 8).
^{#}P < 0.05,
^{###}P < 0.01, compared with the control group
CP: cyclophosphamide;
MN: micronuclei;
NCE: normochromatic erythrocytes;
PCE: polychromatic erythrocytes

As can be seen in Table 10, after 48 hours from the administration of cyclophosphamide, cyclophosphamide may decrease the number of polychromatic erythrocytes and increase that of micronuclei in the blood of the mice, and the WPAF extract in Example 1 has no effect on this phenomenon, indicating that the WPAF extract of the present invention may not influence the anti-cancer effect of cyclophosphamide.

TABLE 11

| | Dose | spleen/body | white blood | |
|---|---|---|---|---|
| | (mg/kg-body | weight | cell | G-CSF |
| | weight) | (%) | (10³/μl) | (pg/ml) |
| Control | 0 | 0.44 ± 0.08 | 5.45 ± 1.89 | 20.1 ± 9.3 |
| CP + H₂O | 0 | 0.34 ± 0.07^{#} | 2.17 ± 0.36^{##} | 227.7 ± 93.5^{##} |
| CP + WPAF extract | 15 | 0.43 ± 0.07* | 3.27 ± 1.13 | 455.0 ± 213.7 |
| CP + WPAF extract | 45 | 0.50 ± 0.06** | 4.33 ± 1.89* | 523.3 ± 229.0* |

All values are mean ± SD (n = 8).
^{##}P < 0.01, compared with the control group.
*P < 0.05,
**P < 0.01, compared with the CP + H₂O group.
CP: cyclophosphamide

As can be seen from Table 11, the WPAF extract of the present invention may improve the reduction of spleen weight and the reduction of the number of leukocytes of the mice caused by cyclophosphamide. In addition, in terms of elevating the G-CSF concentration in the blood of the mice, the WPAF extract of the present invention may promote cyclophosphamide to increase the G-CSF concentration.

The above results show that the WPAF extract of the present invention has no effect on the anti-cancer mechanism or activity of cyclophosphamide, but it can improve the side effect of the reduction of the number of leukocytes caused by cyclophosphamide through stimulating the release of G-CSF.

### Example 6

### Anti-Inflammation Effect of II-AGAF

I. Immune Cell Activation Test

It has been known that some polysaccharides have the activity of activating immune cells, and thus macrophage RAW 264.7 was used to compare the activation effect of II-AGAF and that of lipopolysaccharides (LPS).

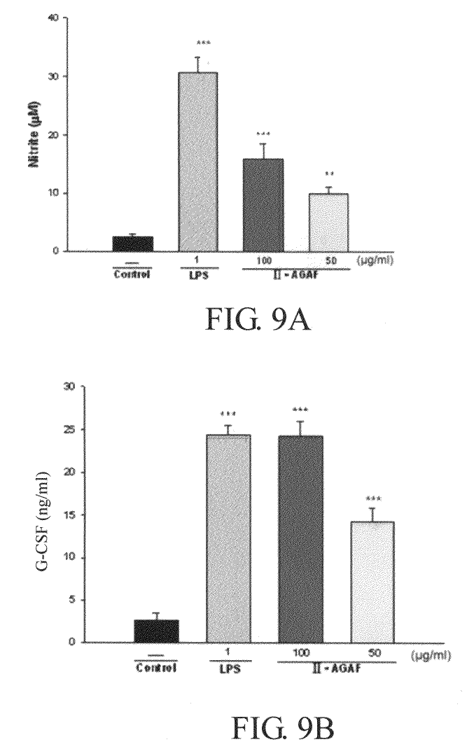

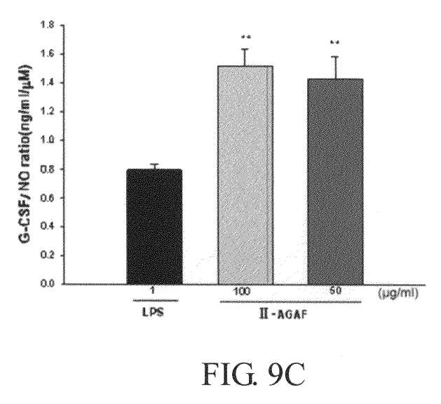

II-AGAF with various concentrations (50 or 100 μg/ml) or LPS (1 μg/ml) was added into a medium (comprising Dulbecco's modified eagle's medium, 10% heat-inactivated fetal bovine serum, 100 U/ml penicillin, and 100 μg/ml streptomycin) containing macrophage RAW 264.7. After the incubation for 24 hours, the broth was centrifuged, and the supernatant was collected. The amount of nitrogen monoxide in the supernatant was determined with a Griess reagent, and G-CSF concentration was measured by ELISA. The results are shown in FIGS. 9A to 9C.

As can be seen in FIGS. 9A to 9C, both II-AGAF and LPS can stimulate macrophages to release nitrogen monoxide and G-CSF. The G-CSF/nitrogen monoxide release ratio of LPS is about 0.8, and that of II-AGAF is about 1.6, indicating that II-AGAF has better selectivity for stimulating the release of G-CSF.

II. Mouse Inflammation Test

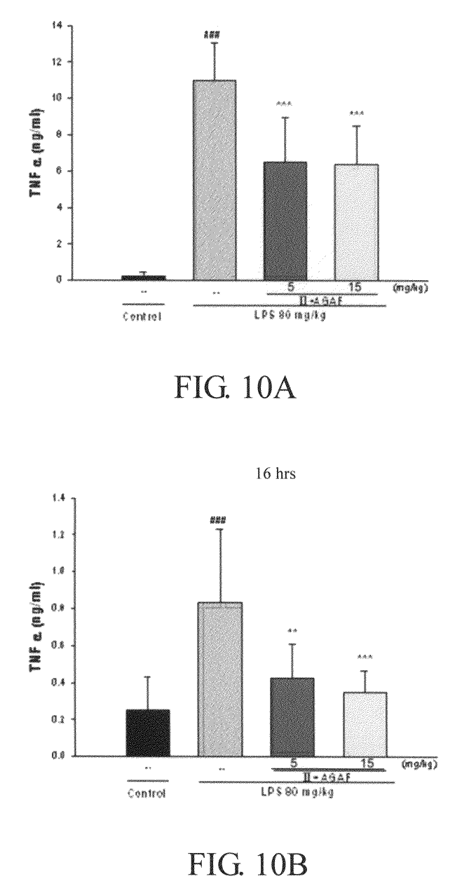

II-AGAF with various dosages (5 or 15 mg/kg-body weight) was administrated to ICR mice through peritoneal cavity injection. After 15 minutes, LPS (80 mg/kg-body weight) was administrated to the ICR mice through peritoneal cavity injection. After 1 and 16 hours, the blood of the ICR mice was collected from orbits, and TNF-α concentration was determined with an enzyme immunoassay agent (eBioscience, Boston, Mass., USA). The results are shown in FIGS. 10A and 10B.

As can be seen from FIGS. 10A and 10B, after 1 and 16 hours from the administration of LPS, II-AGAF obviously decreased the TNF-α concentration in the blood of the mice, indicating that II-AGAF has anti-inflammation effect.

### Example 7

### The WPAF Extract Modulates T Lymphocytes

EL4 cell lines (T cell lines from ATCC (American Tissue Culture Collection), gave by National Health Research Institutes, Taiwan, as a present) were incubated in a DMEM medium (comprising Dulbecco's modified Eagle's medium with 4 mM L-glutamine adjusted to contain 1.5 g/L sodium bicarbonate and 4.5 g/L glucose (90%), and 10% fetal bovine serum), and the WPAF extract in Example 1 (0.8, 4.0, or 20.0 μg/ml) was added into the medium.

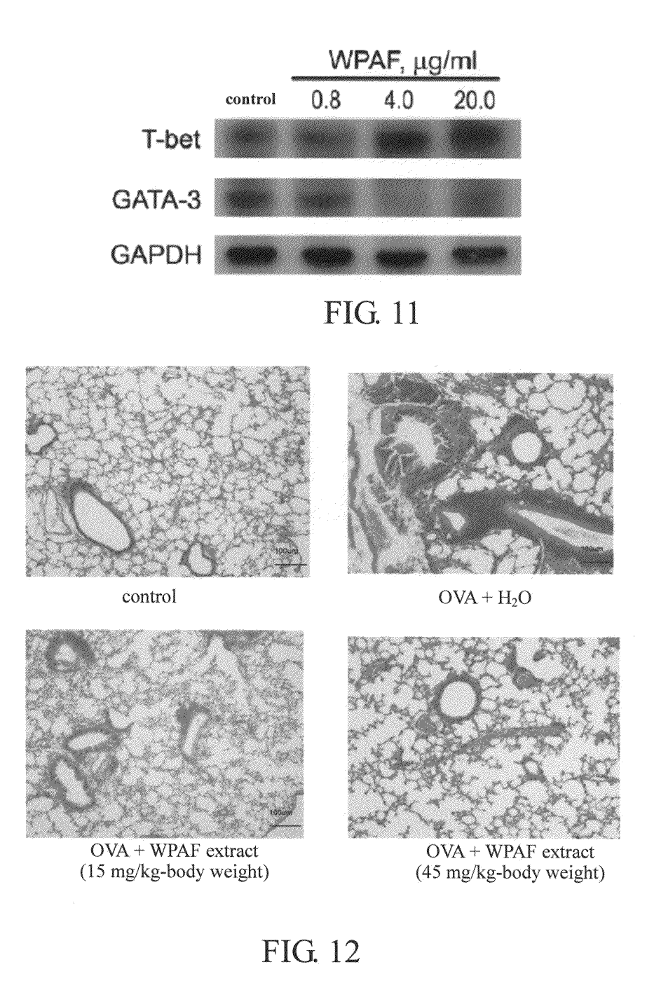

After the incubation for 48 hours, proteins and RNAs in the cells were extracted. The proteins were transferred by western blotting, and antibodies of T-bet, GATA-3, and glyceraldehyde-3-phosphate dehydrogenase (GAPDH) (purchased from Cell Signaling Technology, Inc.) were used to carry out detection, and the result is shown in FIG. 11. Reverse transcription for RNAs was conducted with a MMLV Reverse Transcriptase 1^{st}-Strand cDNA Synthesis Kit (Epicentre Biotechnologies, USA), and specific primers mixed with dUTP and ROX (Protech, Ltd., Taiwan) and a PCR instrument (Smart-Quant Green Real-Time PCR Master) were used to conduct real-time quantitative PCR analysis, and the digitized analysis result is shown in Table 12.

TABLE 12

| | WPAF | WPAF | WPAF |
|---|---|---|---|
| Parameters | (0.8 μg/ml) | (4.0 μg/ml) | (20.0 μg/ml) |
| IL-4 | 0.97 ± 0.13 | 0.56 ± 0.10* | 0.19 ± 0.18** |
| IL-13 | 1.11 ± 0.18 | 0.60 ± 0.18* | 0.32 ± 0.21** |
| IFN-γ | 1.46 ± 0.10 | 5.76 ± 0.14** | 6.51 ± 0.96** |
| GATA-3 | 1.19 ± 0.20 | 0.57 ± 0.14* | 0.52 ± 0.13* |
| T-bet | 1.12 ± 0.16 | 3.48 ± 0.40** | 3.73 ± 0.61** |

All data are corrected with the expression amount of mRNA of GAPDH, and are compared with the control group, and all are mean ± SD (n = 3).
*P < 0.05,
**P < 0.01, compared with the control group.

As shown in Table 12 and FIG. 11, under the stimulation of the WPAF extract (4.0 and 20.0 μg/ml), the protein expression of the transcription factor of EL4 cells, T-bet, obviously increased, and the expression of mRNA of T-bet increased as well. In addition, the mRNA expression of IFN-γ also significantly increased, indicating that the WPAF extract of the present invention may induce the expression of IFN-γ, and thus can stimulate the differentiation of Th1 cells. In another aspect, under the stimulation of the WPAF extract (4.0 and 20.0 μg/ml), the protein expression of the transcription factor GATA-3 obviously decreased, and the mRNA expression thereof decreased as well, and the mRNA expression of IL-4 and IL-13 also decreased remarkably, indicating that the WPAF extract of the present invention may lower the expression of IL-4 and IL-13, and thus has effect of inhibiting the differentiation of Th2 cells.

### Example 8

### The WPAF Extract Improves Asthma

BALB-c male mice of eight weeks old (purchased from National Laboratory Animal Center, Taiwan) were used in the following experiments. The mice were divided into four groups, one of which is the control group, and three of which are the asthma experiment group.

First, the mice were orally administrated with the WPAF extract in Example 1 for a week, and then 0.2 ml albumin (Sigma, Ltd.) was administrated to the mice in the experiment group through peritoneal cavity injection to induce atopic asthma. Hereinafter, the mice with atopic asthma induced by albumin are called “OVA” or “OVA mice.” An aqueous physiological solution of sodium chloride was used as a solvent in the preparation of albumin, and the solution comprises 10 μg grade V albumin (absorbing 40 μg aluminum hydroxide gel (Sigma, Ltd.)). At the 11^{th} to 15^{th} day after the injection of albumin, albumin was dropped into nasal cavities of the mice once a day. After one week, albumin was again dropped into the nasal cavities of the mice, and at the 1^{st}, 6^{th} and 24^{th} hour after the dropping, the resistance force (enhanced pause, Penh) of respiratory tracts of the mice was measured with a breathing apparatus (Buxco MAX II 1320 Modular Unit & Bias Flow Regulator). The measurement of resistance force is conducted by calculating the ratio of peak flow rate of exhalation and inhalation to breathing time. The results are shown in Table 13.

After the experiment proceeded for 48 hours, the mice were sacrificed by anaesthetizing with ether, and collecting the blood from armpit until the death of the mice. Lungs of the mice were washed with 1 ml Hank's Balanced Salt Solutions (Amresco, Solon, Ohio, USA) for three times, and an alveolus rinsing fluid was collected. Then, the lungs of the mice were taken out and bathed in 10 vol % neutral formalin for physiological slice analysis. The physiological slice analysis was conducted with a HE stain method, and cells of the slices were observed. The results are shown in FIG. 12.

Using antibodies on the surface of cells as a marker, and a flow cytometry was used to calculate the number of the cells in the alveolus rinsing fluid, and the cells were classified, and agents used were CD3, CD4, CD8, CD19, CD25, CD45, and CD69 (e-Bioscience, Inc., USA). The amount of cytokines IL-4, IL-5, IL-2, and IFN-γ in the alveolus rinsing fluid was determined with enzyme immunoassay, and assay agents used were purchased from e-Bioscience, Inc., USA. The amount of antibodies IgE, IgG1, and IgG2a in the blood of the mice was also determined by enzyme immunoassay, and assay agents used were purchased from Bethyl Laboratories, USA. The results are shown in Tables 14 to 16.

TABLE 13

| | Dosage | resistance force of respiratory tract | | | |
|---|---|---|---|---|---|
| | (mg/kg) | 0 hr | 1 hr | 6 hr | 24 hr |
| Control | 0 | 0.49 ± 0.06 | 0.52 ± 0.08 | 0.50 ± 0.03 | 0.47 ± 0.05 |
| OVA + H₂O | 0 | 0.49 ± 0.06 | 0.97 ± 0.10^{###} | 0.98 ± 0.11^{###} | 0.61 ± 0.05^{###} |
| OVA + WPAF | 15 | 0.48 ± 0.06 | 0.69 ± 0.09*** | 0.45 ± 0.04*** | 0.51 ± 0.03* |
| extract | | | | | |
| OVA + | 45 | 0.49 ± 0.06 | 0.62 ± 0.08*** | 0.48 ± 0.05*** | 0.52 ± 0.03* |
| WPAF | | | | | |
| extract | | | | | |

All data are mean ± SD (n = 10).
^{###}P < 0.001, compared with the control group.
*P < 0.05,
***P < 0.001, compared with the OVA + H₂O group.

As shown in Table 13, in the analysis of the resistance force of the respiratory tracts, in the OVA+H₂O group, the resistance force of the respiratory tracts of the mice obviously increased at 1^{st} and 6^{th} hour, and slightly relaxed at 24^{th} hour. The WPAF extract of the present invention obviously decreased the resistance force of the respiratory tracts of the mice.

TABLE 14

| | | | WPAF | WPAF |
|---|---|---|---|---|
| | | | extract | extract |
| Parameters | Control | H₂O | (15 mg/kg) | (45 mg/kg) |
| BALF cells (×10⁶) | 0.12 ± 0.01 | 1.32 ± 0.46^{###} | 0.87 ± 0.36* | 0.73 ± 0.30** |
| Lymphocyte (×10⁵) | 0.49 ± 0.06 | 2.07 ± 1.08^{###} | 3.45 ± 1.38* | 3.40 ± 1.27* |
| Macrophage (×10⁵) | 0.51 ± 0.07 | 4.54 ± 2.71^{###} | 3.09 ± 1.78 | 2.04 ± 0.67* |
| Neurophil (×10⁵) | 0.16 ± 0.03 | 1.73 ± 0.88^{###} | 1.50 ± 1.31 | 1.65 ± 1.35 |
| Eosinophil (×10⁵) | 0.00 ± 0.00 | 2.67 ± 0.95^{###} | 0.95 ± 0.94* | 0.55 ± 0.51** |

All data are mean ± SD (n = 10).
^{###}P < 0.001, compared with the control group.
*P < 0.05,
**P < 0.01, compared with the OVA + H₂O group.

As shown in FIG. 12 and Table 14, the WPAF extract of the present invention can inhibit the number of total lung invasive cells (i.e., cells in the alveolus rinsing fluid) in the respiratory tracts of the OVA mice efficiently. For the different kinds of invasive cells, the WPAF extract of the present invention may lower the number of macrophages and eosinophils, but does not influence the number of neurophils. In addition, the WPAF extract of the present invention may also increase the number of lymphocytes. Lymphocytes include T lymphocytes and B lymphocytes, and T lymphocytes have immune modulating activity.

TABLE 15

| | | | WPAF | WPAF |
|---|---|---|---|---|
| | | | extract | extract |
| Parameters | Control | H₂O | (15 mg/kg) | (45 mg/kg) |
| IL-2 (pg/ml) | 44.6 ± 4.8 | 24.6 ± 2.4^{#} | 24.9 ± 14.2 | 32.0 ± 13.7 |
| IL-4 (pg/ml) | 0.0 ± 0.0 | 9.6 ± 23^{###} | 0.9 ± 1.1*** | 2.3 ± 2.4** |
| IL-5 (pg/ml) | 23.1 ± 2.3 | 31.8 ± 7.3^{#} | 15.7 ± 8.6** | 18.2 ± 5.5* |
| IFN-γ (pg/ml) | 54.2 ± 13.2 | 24.3 ± 12.5^{##} | 57.4 ± 28.9*** | 59.4 ± 21.3** |

All data are mean ± SD (n = 10).
^{###}P < 0.001,
^{##}P < 0.01,
^{#}P < 0.05, compared with the control group.
*P < 0.05,
**P < 0.01,
***P < 0.001, compared with the OVA + H₂O group.

As shown in Table 15, the expression of cytokines of the lung invasive cells was observed, and it shows that the WPAF extract of the present invention decreased the amount of IL-4 and IL-5 in the alveolus rinsing fluid, and increased the amount of IFN-γ on the other hand, and slightly increased the amount of IL-2.

Since IFN-γ and IL-2 are cytokines secreted by Th1 cells, and IL-4 and IL-5 are cytokines secreted by Th2 cells, the above result indicates that the WPAF extract of the present invention has immune modulating activity, and may promote the immune response managed by Th1 cells, and meanwhile inhibit the immune response managed by Th2 cells, thereby modulating the immune balance between Th1 cells and Th2 cells.

TABLE 16

| | | | WPAF | WPAF |
|---|---|---|---|---|
| Param- | | | extract | extract |
| eters | Control | H₂O | (15 mg/kg) | (45 mg/kg) |
| IgE (EU) | 0.06 ± 0.01 | 0.29 ± 0.06^{###} | 0.33 ± 0.08 | 0.12 ± 0.05* |
| IgG1 (EU) | 0.07 ± 0.01 | 2.27 ± 0.15^{##} | 2.35 ± 0.23 | 0.19 ± 0.31* |
| IgG2a | 0.02 ± 0.00 | 0.13 ± 0.03^{###} | 0.16 ± 0.04 | 0.55 ± 0.05* |
| (EU) | | | | |

All data are mean ± SD (n = 10).
^{###}P < 0.001,
^{##}P < 0.01, compared with the control group.
*P < 0.05, compared with the OVA + H₂O group.

As shown in Table 16, high dosage (45 mg/kg-body weight) of the WPAF extract of the present invention may decrease the number of specific atopic antibodies (Anti-OVA IgE) and IgG1 antibody modulated by Th2 cells in the serum of the mice. When the mice were administrated with the WPAF extract, the immune system thereof was modulated, and change in the immune balance of Th1/Th2 cells occurred (inclined toward the Th1 cell pathway), which significantly elevated the amount of IgG2a antibody modulated by Th1 cells.

The above result shows that the WPAF extract of the present invention has immune modulating activity, and may modulate the immune balance between Th1 cells and Th2 cells, and stimulate the immune response managed by Th1 cells to increase the amount of IgG2a, and meanwhile inhibit the immune response of Th2 cells to remarkably decrease the amount of atopic antibodies, IgE and IgG1.

FIG. 12 and Tables 13 to 16 indicate that the WPAF extract of the present invention may improve asthma through immune modulation.

### Example 9

### The WPAF Extract Inhibits Colon Cancer

BALB-c female mice of eight weeks old (purchased from National Laboratory Animal Center, Taiwan) were used in the following experiments. The mice were divided into four groups, one of which is the control group, and three of which are the colon cancer experiment group. The experiment group was further divided into a group administrate with water and a group administrated with the WPAF extract in Example 1 (15 or 45 mg/kg-body weight).

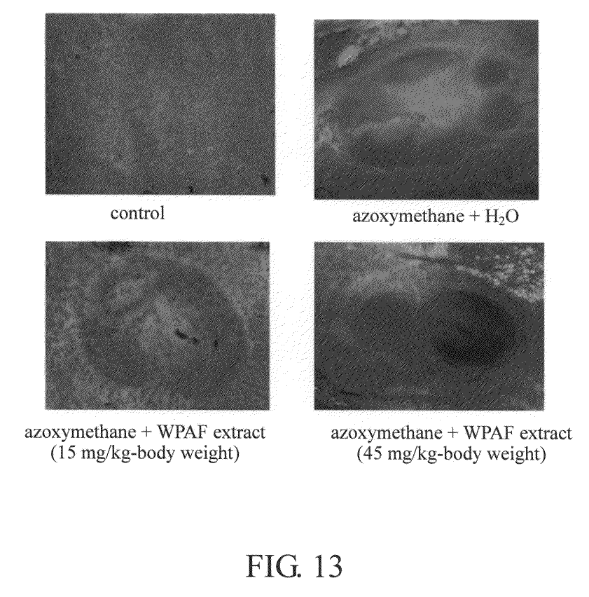

First, the WPAF extract was orally administrated to the mice for two weeks, and then 10 mg/kg-body weight of carcinogen azoxymethane (AOM, Sigma, Ltd) was injected into peritoneal cavities of the mice, and the WPAF extract was orally administrated to the mice continuously. After four months, the mice were sacrificed, and intestines were taken out, and substances in the intestines were washed out with a D-PBS (Dulbecco's Phosphate Buffered Saline) buffer solution. The intestines were then opened by cutting and bathed in a solution of 10 vol % neutral formalin, and were flat fixed. After bathing for one week, the large intestines were stained with methylene blue, and the number of pre-tumor tissues (aberrant crypt foci, ACF) per unit length was calculated. The results are shown in FIG. 13 and Table 17.

Then, mesentery lymph nodes were taken out from large intestines of the mice, and were made into a single cell suspension, and differentiation antigens of a lymphocyte subfamily were analyzed. Subfamilies of immune modulation cells, Treg cells (T regulatory cell, CD4+ and CD 25+), Th1 cells (CD4+ and Tim-3+), and Th2 cells (CD4+ and CD278+), in the mesentery lymph node tissues were analyzed with monoclonal antibodies stained with FITC (fluorescein isothiocyanate) and PE (phycoerythin) and a flow cytometry. The results are shown in Table 18.

Mesentery lymphocytes were incubated in a 24-wells culture plate with a density of 1.0×10⁶/ml, and the cells were stimulated with Con A (concanavalin A). After one to three days, the broth was centrifuged, and the supernatant was collected, and the secretion amount of cytokines, IFN-γ, IL-4, and IL-5, was determined by enzyme immunoassay. The result is shown in Table 19.

TABLE 17

| | Dosage | Aberrant |
|---|---|---|
| | (mg/kg) | crypt foci |
| Control | 0 | 0.23 ± 0.25 |
| azoxymethane + H₂O | 0 | 1.41 ± 0.20^{###} |
| azoxymethane + WPAF extract | 15 | 1.11 ± 0.62 |
| azoxymethane + WPAF extract | 45 | 0.87 ± 0.25* |

All data are mean ± SD (n = 10).
^{###}P < 0.001, compared with the control group.
*P < 0.05, compared with the AOM + H₂O group.

As shown in FIG. 13 and Table 17, when rectum tumors were generated by the induction of azoxymethane, aberrant crypt foci clearly formed, and as the mice were orally administrated with high dosage (45 mg/kg-body weight) of the WPAF extract of the present invention, the number of aberrant crypt foci was efficiently reduced.

TABLE 18

azoxymethane

| | | | WPAF | WPAF |
|---|---|---|---|---|
| Parameters | Control | H₂O | (15 mg/kg) | (45 mg/kg) |
| T4 | 55.1 ± 3.0 | 55.8 ± 4.2 | 50.0 ± 6.1 | 54.3 ± 3.5 |
| (CD4+, CD3+) | | | | |
| T8 | 21.6 ± 2.2. | 25.3 ± 2.4 | 23.5 ± 1.9 | 25.2 ± 1.8 |
| (CD8+, CD3+) | | | | |
| T | 73.5 ± 2.9 | 77.0 ± 3.5 | 72.3 ± 4.3 | 76.8 ± 3.5 |
| (CD3+, CD45+ | | | | |
| B | 23.5 ± 4.2 | 22.3 ± 4.5 | 27.0 ± 3.4 | 21.2 ± 2.6 |
| (CD19+, | | | | |
| CD45+) | | | | |
| Treg | 7.2 ± 0.5 | 8.2 ± 0.2^{#} | 6.8 ± 0.4** | 7.7 ± 0.9 |
| (CD4+, | | | | |
| CD25+) | | | | |
| Th1 | 4.5 ± 0.2 | 3.2 ± 0.3^{#} | 4.6 ± 0.4* | 4.8 ± 0.4* |
| (CD4+, | | | | |
| Tim-3+) | | | | |
| Th2 | 4.0 ± 0.8 | 5.2 ± 1.1^{#} | 3.7 ± 1.1* | 3.8 ± 0.9* |
| (CD4+, | | | | |
| CD278+) | | | | |

All data are mean ± SD (n = 10), and the unit is %.
^{#}P < 0.05, compared with the control group.
*P < 0.05,
**P < 0.01, compared with the OVA + H₂O group.

As shown in Table 18, when large intestine tumors of the mice were generated by the induction of azoxymethane, the number of Treg cells clearly increased, and as the mice were orally administrated with low dosage (15 mg/kg-body weight) of the WPAF extract of the present invention, the ratio of Treg cells was reduced, indicating that the WPAF extract has activity of inhibiting tumor cells.

In another aspect, it was discovered in the composition analysis of the subfamilies of Th1 cells and Th2 cells that, as rectum tumors were generated by the induction of azoxymethane, the ratio of Th1 cells (CD4+ and Tim-3+) decreased obviously, and after the WPAF extract of the present invention was administrated to the mice, the ratio increased remarkably. In addition, as rectum tumors were generated by the induction of azoxymethane, the ratio of Th2 cells (CD4+ and CD278+) increased significantly. As stated above, it is known that the elevation of the immune response of Th2 cells promotes tumor formation induced by azoxymethane. Because after the mice were administrated with the WPAF extract of the present invention, the ratio of Th2 cells decreased remarkably, it indicates that the WPAF extract of the present invention may inhibit the tumor formation through lowering the immune response of Th2 cells.

TABLE 19

| | Dosage | IL-4 | IL-5 | IFN-γ |
|---|---|---|---|---|
| Treatments | (mg/kg) | (pg/ml) | (pg/ml) | (pg/ml) |
| Control | 0 | 20.6 ± 7.4 | 18.2 ± 9.2 | 1.3 ± 0.2 |
| azoxymethane + H₂O | 0 | 39.0 ± 14.9^{#} | 31.3 ± 15.3^{#} | 1.0 ± 0.2^{#} |
| azoxymethane + | 15 | 29.8 ± 11.5 | 27.1 ± 14.0 | 1.2 ± 0.4 |
| WPAF extract | | | | |
| azoxymethane + | 45 | 29.6 ± 11.0 | 26.4 ± 14.6 | 1.3 ± 0.2* |
| WPAF extract | | | | |

All data are mean ± SD (n = 10).
^{#}P < 0.05, compared with the control group.
*P < 0.05, compared with the OVA + H₂O group.

As shown in Table 19, the WPAF extract of the present invention may increase IFN-γ secretion and inhibit IL-4 and IL-5 secretion of the mice with tumors induced by azoxymethane.

The above experiments show that the WPAF extract of the present invention can modulate the immune balance between Th1 cells and Th2 cells, and promote the immune response managed by Th1 cells, and meanwhile inhibit the immune response of Th2 cells, so as to inhibit colon cancer through immune modulation.

### Example 10

### Determination of the Active Component of the WPAF Extract

I. Prebiotics Effect of II-AGAF

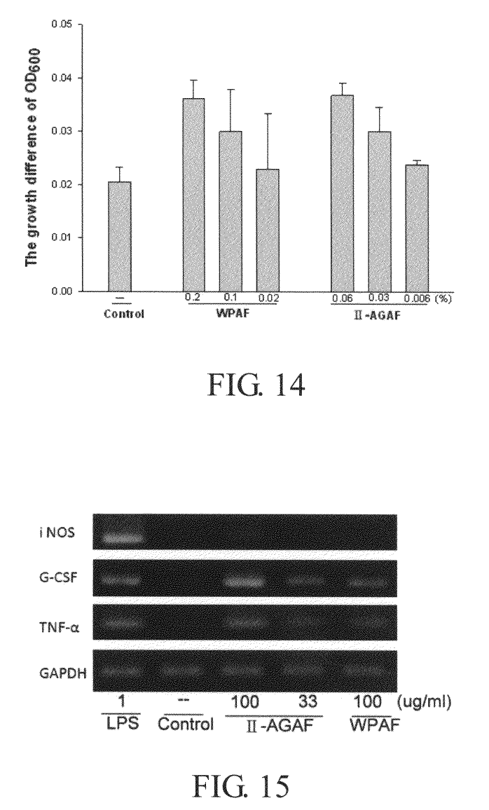

As can be seen in Table 1 of Example 1, the WPAF extract in Example 1 contains 33.4 wt % of II-AGAF. The WPAF extract in Example 1 and II-AGAF were used to carry out a prebiotics assay, respectively, and Bifidobacterium breve was used as an advantageous bateria. The amount of II-AGAF used in the assay corresponds to the II-AGAF content in the WPAF extract (for instance, as the dosage of the WPAF extract is 0.6 g, then that of II-AGAF in the comparison group is about 0.2 g). The results are shown in FIG. 14. As shown in FIG. 14, the prebiotics effect provided by the WPAF extract of the present invention and II-AGAF with the corresponding amount is equal.

II. Activating Immune Cells Effect of II-AGAF

Similar to the above prebiotics assay, the WPAF extract in Example 1 and II-AGAF with the corresponding amount were added into a medium containing macrophage RAW 264.7 respectively. After the incubation for 24 hours, mRNAs were extracted, and RT-PCR was conducted to analyze the expression of nitrogen monoxide synthetase, G-CSF, and TNF-α. As shown in FIG. 15, the effect of activating immune cells provided by the WPAF extract of the present invention and II-AGAF with the corresponding amount is equal.

From the above results, it can be inferred that the main active component of the WPAF extract of the present invention is II-AGAF. The other components in the WPAF extract, like starch, do not provide specific physiological activity. Without being restricted by theory, it is believed that after starch enters the digestive tract, it is degraded into glucose by amylase, and thus, it cannot provide specific physiological activity.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. An Anoectochilus spp. polysaccharide extract for stimulating the growth of advantageous bacteria, stimulating the release of granulocyte colony-stimulating factor (G-CSF), modulating T helper cell type I (Th1 cell), and/or modulating T helper cell type II (Th2 cell), comprising a type II arabinogalactan of Anoectochilus spp. and having an average molecular weight of about 40 to about 70 kilodaltons.

2. The extract as claimed in claim 1, wherein the advantageous bacteria belong to Bifidobacterium genus.

3. The extract as claimed in claim 1, wherein the advantageous bacteria are Bifidobacterium breve.

4. The extract as claimed in claim 1, wherein the extract has an average molecular weight ranging from about 50 to about 60 kilodaltons, and the type II arabinogalactan has an average molecular weight ranging from about 15 to about 45 kilodaltons.

5. The extract as claimed in claim 1, wherein the type II arabinogalactan has an average molecular weight ranging from about 25 to about 35 kilodaltons.

6. The extract as claimed in claim 1, which is in the form of an aqueous solution.

7. The extract as claimed in claim 1, wherein the Anoectochilus spp. is Anoectochilus formosanus Hayata.

8. The extract as claimed in claim 1, wherein the type II arabinogalactan is in an amount of about 20 wt % to about 50 wt %, based on the dry weight of the extract.

9. The extract as claimed in claim 1, wherein the type II arabinogalactan is in an amount of about 30 wt % to about 40 wt %, based on the dry weight of the extract.

10. The extract as claimed in claim 1, which is useful for increasing an amount of fatty acids in an intestine, stimulating calcium absorption, anti-osteoporosis, anti-inflammation, inhibiting a decrease of leukocytes, anti-allergy, improving asthma, inhibiting colon cancer, and/or modulating immunological functions.

11. A method of the preparation of the Anoectochilus spp. polysaccharide extract as claimed in claim 1, comprising:
d) extracting Anoectochilus spp. with water to obtain a water-soluble Anoectochilus spp. extract;
e) de-fatting the Anoectochilus spp. extract and then collecting an aqueous extract; and
f) adding ethanol to the aqueous extract and then collecting a precipitate, wherein the amount of ethanol is about 65 vol % to about 85 vol %, based on the total volume of the aqueous extract and ethanol.

12. The method as claimed in claim 11, wherein in step b), the de-fatting step is conducted by adding about 15 vol % to about 35 vol % of ethyl acetate to the Anoectochilus spp. extract, based on the volume of the Anoectochilus spp. extract.

13. The method as claimed in claim 11, wherein in step b), the de-fatting step is conducted by adding about 20 vol % to about 30 vol % of ethyl acetate to the Anoectochilus spp. extract, based on the volume of the Anoectochilus spp. extract.

14. The method as claimed in claim 11, wherein the amount of ethanol is about 70 vol % to about 80 vol %, based on the total volume of the aqueous extract and ethanol.

15. The method as claimed in claim 11, further comprising dissolving the precipitate collected in step c) in water.

16. A method for stimulating the growth of advantageous bacteria, stimulating the release of granulocyte colony-stimulating factor (G-CSF), modulating T helper cell type (Th1 cell), and/or modulating T helper cell type II (Th2 cell) in a mammal, comprising administrating an effective amount of the extract as claimed in claim 1 to the mammal.

17. The method as claimed in claim 16, which is for increasing an amount of fatty acids in an intestine, stimulating calcium absorption, anti-osteoporosis, anti-inflammation, inhibiting a decrease of leukocytes, anti-allergy, improving asthma, inhibiting colon cancer, and/or modulating immunological functions.

18. The method as claimed in claim 16, wherein the extract is administrated as a medicament.

19. The method as claimed in claim 16, wherein the amount of the extract administrated to the mammal, calculated as the type II arabinogalactan, is about 2 mg/kg-body weight to about 25 mg/kg-body weight per day.

20. The method as claimed in claim 16, wherein the amount of the extract administrated to the mammal, calculated as the type II arabinogalactan, is about 3 mg/kg-body weight to about 20 mg/kg-body weight per day.
